(19) United States
(12) Patent Application Publication
(10) Pub. No.: US 2020/0146618 A1
(43) Pub. Date: May 14, 2020

(54) DEVICE WITH A DETECTION UNIT FOR THE POSITION AND ORIENTATION OF A FIRST LIMB OF A USER

(71) Applicant: Psii.rehab GmbH, Bad Ischl (AT)

(72) Inventor: Georg Teufl, Bad Ischl (AT)

(21) Appl. No.: 16/619,384

(22) PCT Filed: May 17, 2018
(86) PCT No.: PCT/AT2018/050011

§ 371 Date: Dec. 4, 2019

(30) Foreign Application Priority Data
Jun. 7, 2017 (AT) A 60047/2017

(51) Int. Cl. A61B 5/00; A61B 5/11; A61B 5/0488; H04N 13/344; G06F 3/01; G09B 19/00

(52) U.S. Cl. CPC A61B 5/486; A61B 5/1114; A61B 5/0488; G09B 19/003; H04N 13/344; G06F 3/017; G06F 3/015; A61B 5/744

(57) ABSTRACT

The invention relates to a device comprising a detection unit (1) for the position and orientation of a first limb of a user (3) and a display device (4) for displaying a second limb (5). In order to design a device of the type described above so as to expand the range of motion for the two limbs of the user and at the same time allow interaction between the two limbs and between one of the limbs and a virtual three-dimensional object, it is proposed that the display device is designed stereoscopically and has, with the detection unit, a common reference plane as a reference system, in that, for display via the stereoscopic display device, a first generator is provided for a virtual second limb which is mirrored about the reference plane with respect to the position and orientation of the first limb, and a second generator is provided for a three-dimensional interaction object for display at a predetermined position and orientation with respect to the reference plane, and in that the device has a collision detection unit for outputting a signal upon detection of a collision between the virtual second limb and the virtual three-dimensional interaction object.

### FIELD OF THE INVENTION

The invention relates to a device comprising a detection unit for the position and orientation of a first limb of a user and a display device for displaying a second limb.

### DESCRIPTION OF THE PRIOR ART

Devices are known for use in mirror therapy (US 20150313793 A1) which record the position and orientation of a first limb using a detection unit, in this case a camera, and display a virtual second limb created by mirroring the recorded image on a display device. However, the disadvantage is that the possible range of movement is severely restricted because the second limb of the user must always be below the display device or the virtual second limb must always be within the display device in order to maintain the effect of mirror therapy. In particular, this technical solution does not provide the possibility to interact with both limbs.

### SUMMARY OF THE INVENTION

The invention is thus based on the object of designing a device of the type described above in such a way that the space of movement for the two limbs of the user is extended and at the same time an interaction between the two limbs but also between one of the limbs and a virtual three-dimensional object is made possible.

The invention solves the set object in that the display device is stereoscopically formed and has a common reference plane as reference system with the detection unit, in that, for display via the stereoscopic display device, a first generator is provided for a virtual second limb which is mirrored about the reference plane with respect to the position and orientation of the first limb, and a second generator is provided for a three-dimensional interaction object for display at a predetermined position and orientation with respect to the reference plane, and in that the device has a collision detection unit for outputting a signal upon detection of a collision between the virtual second limb and the virtual three-dimensional interaction object. As a result of these measures, the user is largely free to move the first limb as long as it is within the detection range of the detection unit. Particularly advantageous in this context is that the display device and the detection unit share a common reference plane as a reference system, which preferably coincides with the sagittal plane of the user or more precisely the head of the user. This can be achieved, for example, by the stereoscopic display device being worn by the user in the form of spectacles and thus the median plane of the stereoscopic display device coinciding with the sagittal plane of the user and thus with the common reference plane, and by the detection unit on this display device being arranged centrally in the reference plane. It is understood that the detection unit may also be arranged differently from this reference plane, provided that the relative position of the detection unit to the reference plane is known as the reference system. The same applies in principle to the stereoscopic display device, so that stationary stereoscopic display devices are also conceivable, but a particular advantage arises from the fact that the stereoscopic display device as a pair of spectacles itself with its median plane essentially coincides with the sagittal plane of the user. The stereoscopic display device may be such that the user's first limb remains visible through the display device, while the user's second limb is superimposed by a virtual second limb, so that from the user's perspective the second limb is no longer visible in the real world. However, both the first and the second limb of the user can be displayed in a common virtual space via the stereoscopic display device. In order to train the fine motor skills of the user in particular, a second generator is also provided for a three-dimensional interaction object, which is displayed to the user via the stereoscopic display device at a predetermined position and orientation relative to the reference plane. In order to give the user feedback on the interaction with the three-dimensional interaction object, a collision detection unit is also provided to output a signal upon detection of the collision between the virtual second limb and the virtual three-dimensional interaction object. The collision detection unit can also be extended to detect collisions between the first limb and the second limb or the first limb and the virtual three-dimensional interaction object. The signal emitted by the detection unit can be optically displayed to the user directly via the stereoscopic display, or converted into an auditory or tactile signal for the user. For the sake of completeness, it should be noted that a limb is defined as a column of limbs and/or tip of a limb, i.e. upper or lower arm, thigh or lower leg and/or hand or foot.

So far, it has been assumed that the user cannot use the second limb or can only use it to a very limited extent. However, if measurable muscle activity is present, sensors may be provided for detecting the electromyogram of muscle groups of the second limb of the user, which are connected via a control unit to the first generator for changing the position and orientation of the virtual second limb as a function of the action potentials detected. The position and orientation of the generated, mirrored virtual second limb can thus be changed on the basis of the mirrored model as a function of the recorded electromyogram, giving the user the impression that the virtual second limb is actually moving in virtual space with the muscle groups he is controlling. This has the advantage over the known devices that the mirror therapy can be extended by a direct feedback for the user, so that preliminary exercises can be carried out to control, for example, a prosthesis or the like. In addition, the posture of the limb itself can also be changed on the basis of the action potentials recorded, especially if the model of the virtual second limb is designed as a kinematic model of individual limb parts.

In order to create a realistic impression for the user, the collision detection unit can be connected to the first generator to change the position and orientation of the virtual second limb depending on the detected collisions. As a result, not only can the virtual three-dimensional interaction object with the two limbs be moved or shifted, but the virtual second limb can also be shifted by the first limb or the virtual object by an imaginary application of force, unless, for example, an imaginary counter-pressure is generated by a corresponding action potential as described above.

To provide the user with a complete virtual interactive experience, it is proposed that the detection unit comprises a depth sensor to which the first generator is connected to generate a virtual model of the second limb at a position and orientation of the first limb mirrored about the reference plane as a virtual second limb. In this case, neither the first nor the second limb is visible to the user, but only virtual models of these first and second limbs, which are displayed in a virtual space via the stereoscopic display device. The fact that the first limb is completely known in its spatial position by the depth sensor in this case results in considerably more interaction possibilities. For example, in combination with the recording of the electromyogram of muscle groups of the second limb, an interaction can be displayed for the user in which both limbs are at different depth distances, which is impossible with a previously known mirror therapy.

In order to avoid complex menu navigation, the first generator can have an interaction element memory and be connected to a gesture recognition unit for selecting one of the interaction elements from the interaction element memory as a function of the gesture recognized. As a result of these measures, depending on the posture of the first limb, a virtual three-dimensional interaction element corresponding to this gesture, e.g. a pincer grip, can be displayed to the user, which is best suited, for example, for the posture of the limb for an interaction.

The subject matter of the invention also relates to a method for operating the device according to the invention, wherein the position and orientation of a first limb of a user relative to a reference plane corresponding to the sagittal plane of the user is detected and fed to a first generator which generates a virtual second limb mirrored in position and orientation of the first limb about the reference plane for display via a stereoscopic display device, wherein a second generator generates a three-dimensional interaction object at a predetermined position and orientation relative to the reference plane, and wherein a collision detection unit outputs a signal upon detection of a collision between the virtual second limb and the virtual three-dimensional interaction object.

In this context, particularly advantageous operating characteristics result when the second generator generates the three-dimensional interaction element only when the first and/or second limb falls below a specified minimum distance to the reference plane. It is also possible to generate the three-dimensional interaction element only after a collision between the first and second limb.

As mentioned above, a gesture recognition unit may be provided that recognizes gestures performed with the first or second limb and the second generator selects a three-dimensional interaction element from an interaction element memory for display depending on the recognized gesture.

### BRIEF DESCRIPTION OF THE INVENTION

The drawings show the subject matter of the invention by way of example, wherein:

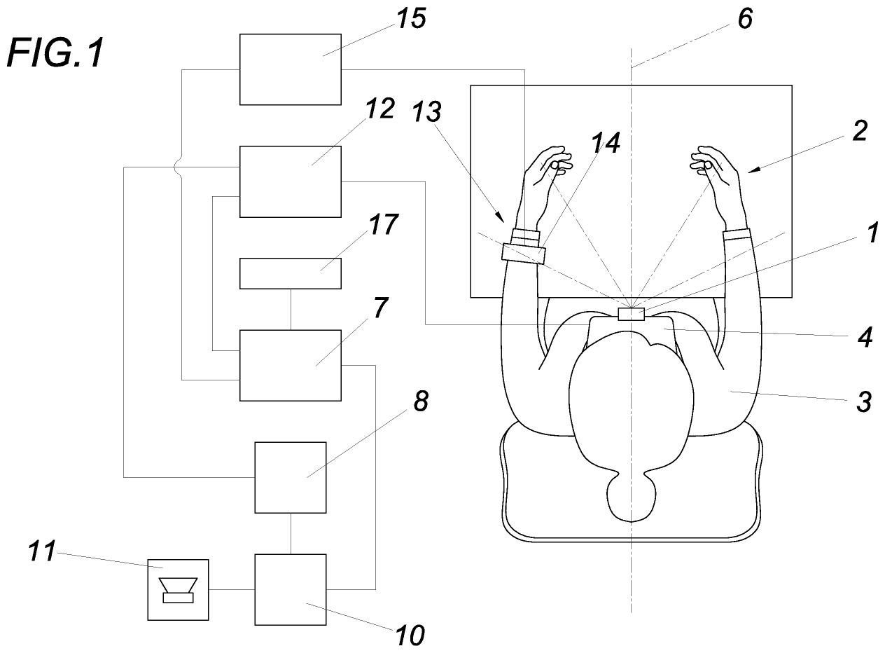

FIG. 1 shows a device according to the invention operated by a user in a schematic plan view, and

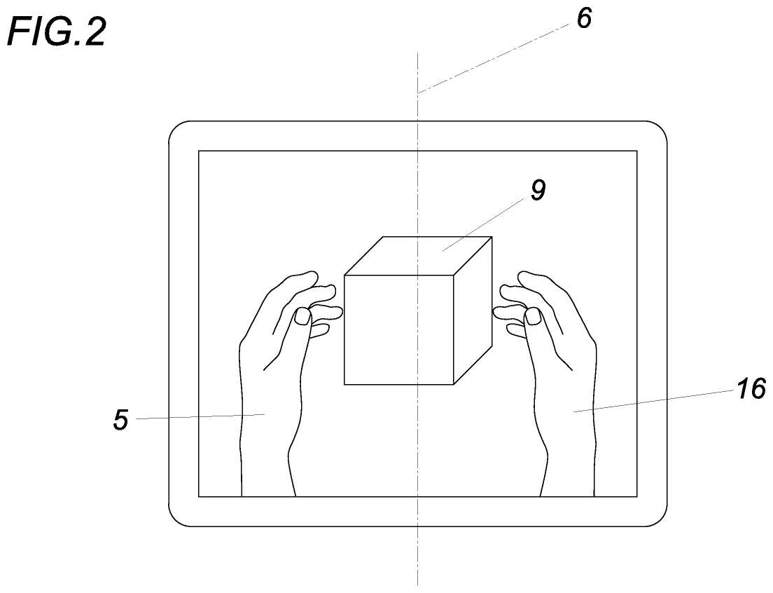

FIG. 2 shows a schematic view of the display presented to the user in a two-dimensional simplified representation.

### DESCRIPTION OF THE PREFERRED EMBODIMENTS

A device according to the invention comprises a detection unit 1 for the position and orientation of a first limb 2 of a user 3 and a stereoscopic display device 4 for displaying a virtual second limb 5. The detection unit 1 and the stereoscopic display device 4 have a common reference plane 6 as a reference system. The virtual second limb 5 is generated by a first generator 7 connected to the detection unit 1 for this purpose. In addition, a second generator 8 is planned for a three-dimensional interaction object 9. To detect a collision between the virtual second limb 5 and the virtual three-dimensional interaction object 9, a collision detection unit 10 is provided, which outputs a signal via a loudspeaker 11 when a collision is detected. For this purpose, the collision detection unit 10 is connected to both the first generator 7 and the second generator 8. A display control unit 12 is provided for the stereoscopic display device 4, which processes the output signals of the first generator 7 and the second generator 8 and thus controls the stereoscopic display device 4.

In order to be able to change the position and orientation of the virtual second limb 5 as a function of the action potentials of individual muscle groups of the real second limb 13, the device according to invention comprises, in a preferred embodiment, sensors 14 for detecting the electromyogram of muscle groups of the second limb 13, wherein the sensors 14 are connected via a control unit 15 to the first generator 7 for changing the position and orientation of the virtual second limb 5 as a function of the action potentials detected via the sensors 14.

The collision detection unit 10 can also be connected to the first generator 7 to change the position and orientation of the virtual second limb 5 as a function of detected collisions.

In the embodiment example shown, detection unit 1 also includes a depth sensor which is not shown in detail and which is also connected to the first generator 7. This generates not only a virtual model of the second limb 5, but also a virtual model of the first limb 16, so that in the stereoscopic view the user has a complete virtual space with a first virtual limb 16, a second virtual limb 5 and a virtual three-dimensional interaction element 9.

To select an interaction element from an unspecified interaction element memory in response to a gesture, the first generator 7 is connected to a gesture recognition unit 17 which recognizes the gesture performed by users with the respective limb either via the detection unit 1 or already via the generated models of the first or second limb 5, 16, whereupon the first generator selects a corresponding virtual interaction element 9 in response to the recognized gesture.

1. A device comprising:
a detection unit detecting a position and orientation of a first limb of a user; and
a display device displaying a second limb; wherein the display device is of stereoscopic design and has a reference plane as a reference system in common with the detection unit;
wherein a first generator generating a virtual second limb mirrored about the reference plane with respect to the position and orientation of the first limb and a second generator generating a three-dimensional interaction object displayed at a predetermined position and orientation with respect to the reference plane via the stereoscopic display device, and
wherein the device has a collision detection unit outputting a signal upon detection of a collision between the virtual second limb and the virtual three-dimensional interaction object.

2. A device according to claim 1, wherein sensors detect an electromyogram of muscle groups of the second limb of the user, and said sensors are connected via a control unit to the first generator and change the position and orientation of the virtual second limb as a function of detected action potentials.

3. A device according to claim 1 wherein the collision detection unit changes the position and orientation of the virtual second limb as a function of detected collisions and is connected to the first generator.

4. A device according to claim 1, wherein the detection unit comprises a depth sensor to which the first generator generating a virtual model of the second limb at a position and orientation of the first limb mirrored about the reference plane as the virtual second limb is connected.

5. A device according to claim 1, wherein the first generator has an interaction element memory and is connected to a gesture recognition unit selecting one of a plurality of interaction elements from the interaction element memory as a function of a gesture when recognized.

6. A method for operating a device according to claim 1, said method comprising
detecting the position and orientation of the first limb of the user relative to the reference plane corresponding to a sagittal plane of the user; and
feeding the position and orientation of the first limb to the first generator; and
generating with the first generator the virtual second limb, mirrored with respect to the position and orientation of the first limb about the reference plane;
displaying display the virtual second limb via a stereoscopic display device;
generating with the second generator the three-dimensional interaction object at a predetermined position and orientation relative to the reference plane; and
outputting from the collision detection unit a signal upon detection of a collision between the virtual second limb and the virtual three-dimensional interaction object.

7. A method according to claim 6, wherein the second generator generates the three-dimensional interaction element only if the first or second limb falls below a predetermined minimum distance to the reference plane.

8. A method according to claim 6, wherein a gesture recognition unit recognizes a gesture performed with the first or second limb and the second generator selects said three-dimensional interaction element from an interaction element memory as a function of the recognized gesture.

9. A device according to claim 2 wherein the collision detection unit changes the position and orientation of the virtual second limb as a function of detected collisions and is connected to the first generator.

10. A device according to claim 9, wherein the detection unit comprises a depth sensor to which the first generator generating a virtual model of the second limb at a position and orientation of the first limb mirrored about the reference plane as the virtual second limb is connected.

11. A device according to claim 2, wherein the detection unit comprises a depth sensor to which the first generator generating a virtual model of the second limb at a position and orientation of the first limb mirrored about the reference plane as the virtual second limb is connected.

12. A device according to claim 3, wherein the detection unit comprises a depth sensor to which the first generator generating a virtual model of the second limb at a position and orientation of the first limb mirrored about the reference plane as the virtual second limb is connected.

13. A device according to claim 2, wherein the first generator has an interaction element memory and is connected to a gesture recognition unit selecting one of a plurality of interaction elements from the interaction element memory as a function of a gesture when recognized.

14. A device according to claim 3, wherein the first generator has an interaction element memory and is connected to a gesture recognition unit selecting one of a plurality of interaction elements from the interaction element memory as a function of a gesture when recognized.

15. A device according to claim 4, wherein the first generator has an interaction element memory and is connected to a gesture recognition unit selecting one of a plurality of interaction elements from the interaction element memory as a function of a gesture when recognized.

16. A device according to claim 9, wherein the first generator has an interaction element memory and is connected to a gesture recognition unit selecting one of a plurality of interaction elements from the interaction element memory as a function of a gesture when recognized.

17. A device according to claim 10, wherein the first generator has an interaction element memory and is connected to a gesture recognition unit selecting one of a plurality of interaction elements from the interaction element memory as a function of a gesture when recognized.
